(19) United States
(12) Patent Application Publication
(10) Pub. No.: US 2013/0267532 A1
(43) Pub. Date: Oct. 10, 2013

(54) CYCLIC AMIDE COMPOUNDS AND THEIR USE IN THE TREATMENT OF DISEASE

(71) Applicants: Shinya Tosaki, Osaka-shi, Osaka (JP); Seiji Hori, Osaka (JP)

(72) Inventors: Shinya Tosaki, Osaka-shi, Osaka (JP); Seiji Hori, Osaka (JP)

(21) Appl. No.: 13/885,727

(22) PCT Filed: Nov. 18, 2011
(86) PCT No.: PCT/JP2011/077274

§ 371 Date: Jun. 14, 2013

Related U.S. Application Data
(60) Provisional application No. 61/415,700, filed on Nov. 19, 2010.

(51) Int. Cl. C07D 403/10

(52) U.S. Cl. CPC C07D 403/10
USPC 514/252.14; 544/295

(57) ABSTRACT

The invention concerns compounds of Formula (I): (I) and pharmaceutically acceptable salts thereof, wherein n, R¹ and R² are as defined in the description. The present invention also relates to processes for the preparation of such compounds, novel intermediates useful in the preparation of such compounds, pharmaceutical compositions containing them and their use in the treatment of disease, for example cancer.

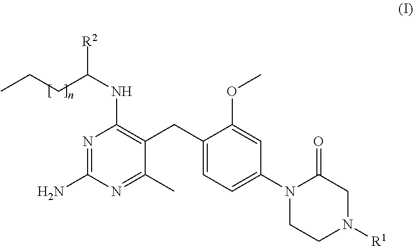

### FIELD OF THE INVENTION

The present invention relates to novel cyclic amide compounds and, more particularly, to novel cyclic amide compounds that act as TLR7 agonists. This invention also relates to methods for the preparation of such compounds and novel intermediates in the preparation thereof, to pharmaceutical compositions containing such compounds, to the use of such compounds in the preparation of medicaments, and to the use of such compounds in the treatment of conditions mediated by TLR7, such as allergic diseases, autoimmune diseases, viral diseases and, in particular, cancer.

### BACKGROUND OF THE INVENTION

Toll-like receptors (TLRs) are expressed on a variety of immune cells, including macrophages and dendritic cells (DCs). TLRs recognise molecular motifs on pathogens called pathogen-associated molecular patterns (PAMPs). To date, 13 TLRs have been identified in man, these include TLRs 1, 2, 4, 5 and 6, which are confined to the cell surface and TLRs 3, 7, 8 and 9 which are expressed in endosomes. Different TLRs recognise different pathogen-derived ligands, for example TLRs 2 (bacterial lipoproteins), 3 (double-stranded RNA/poly (I:C)), 4 (lipopolysaccharides), 5 (flagellin), 7 (single-stranded RNA) and 9 (CpG-containing DNA). Ligation of TLRs on antigen-presenting cells, such as DCs, leads to production of proinflammatory cytokines, DC maturation and priming of the adaptive immune system. TLR7 and TLR9 are expressed by plasmacytoid dendritic cells (pDCs) and ligand recognition leads to the secretion of interferon-α (INF-α). Preclinical studies investigating the effects of activation of TLRs, using bacterial or viral components, dosed as monotherapy and/or combined with anti-tumor agents, have shown tumour growth inhibition in a variety of murine tumour models.

Several small molecule TLR7 agonists have been described, including the imidazoquinoline, imiquimod, which has been used to treat a number of dermatological conditions e.g. genital warts, molluscum contagiosum and melanoma. In the case of melanoma, topically applied imiquimod (Aldara™, Graceway Pharmaceuticals, Bristol, Tenn.) demonstrated therapeutic responses in cutaneous metastatic melanoma and lentigo maligna and has been approved for the treatment of superficial basal cell carcinoma (BCC). Preclinical and clinical studies indicate that imiquimod is likely to function through the induction of type 1 IFN and IFN-inducible genes, which in turn can have direct effects on tumour cell growth and/or harness components of the adaptive immune system. 852A is another imidazoquinoline, which unlike imiquimod, is suitable for systemic administration. Currently 852A is in phase II clinical trials in a number of cancer indications, including melanoma.

Nevertheless, there remains a need for further TLR7 agonists which are more effective in the treatment of disease, for example cancer, by reason of their superior potency and/or advantageous physical properties (for example, higher permeability, and/or lower plasma protein binding) and/or favourable toxicity profiles and/or favourable metabolic profiles in comparison with other known TLR7 agonists, for example 852A.

### SUMMARY OF THE INVENTION

As now demonstrated herein, the cyclic amide compounds of the present invention are capable of activating TLR7 in vitro. As a consequence of this activity, the cyclic amide compounds of the present invention are expected to have value in the prevention or treatment of human disease, for example cancer, either as a monotherapy or in combination with other chemotherapeutic agents or radiotherapy regimens.

As a further feature of the invention, compounds of the invention have surprisingly advantageous selectivity for TLR7 over TLR8. TLR7 and TLR8 differ in their cellular expression and as a result stimulation with selective agonists induces different cytokine profiles. TLR8 stimulation (either as a TLR8 selective agonist or a TLR7/8 dual agonist) results in enhanced levels of pro-inflammatory cytokines including TNFα, IL-1β and IL-6 (Gorden et al (2005) J. Immunol. 174, 1259-1268). Conversely, TLR8 stimulation results in lower levels of IFNα. Therefore, a TLR7 selective agonist would favour induction of IFNα, which is important in suppression of Th2 cytokines (Huber et al (2010) J. Immunol. 185; 813-817) that are elevated in allergic disease. In addition, by making compounds selective for TLR7 compared to TLR8 the induction of proinflammatory cytokines would be reduced thus avoiding inflammatory responses in man.

As a further feature of the invention, some compounds of the invention also have a surprisingly advantageous PAMPA (Parallel Artificial Membrane Permeability Assay) profile. Compounds that have low value on PAMPA are disfavored because low permeability is implicated in the deficiency of oral administration of compounds.

As a further feature of the invention, certain compounds of the invention also have a surprisingly advantageous hERG profile. Compounds that have significant activities against the hERG ion channel are relevant to inducing QT prolongation and are disfavored because such activity is implicated in the development of Torsades de Pointes and cardiac death.

According to a first aspect of the present invention, there is therefore provided a compound of Formula (I), or a pharmaceutically acceptable salt thereof:

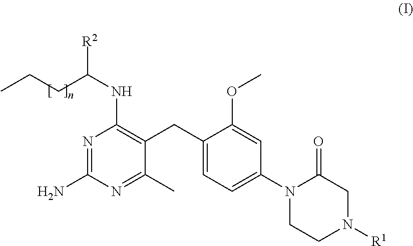

wherein:
n is 1 or 2;
R¹ is selected from hydrogen, C₁₋₄alkyl, C₁₋₃alkoxyC₁₋₄alkyl, hydroxyC₂₋₄alkyl and (R^{X})(R^{Y})N—C₂₋₄alkyl, wherein R^{X} and R^{Y} each independently represent hydrogen or C₁₋₃alkyl; and
R² is hydrogen, hydroxymethyl or 2-hydroxyethyl.

It is to be understood that, insofar as certain of the compounds of Formula (I) defined above may exist in optically active or racemic forms by virtue of one or more asymmetric carbon atoms, the invention includes in its definition any such optically active or racemic form which possesses the above-mentioned activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

It is to be understood that certain compounds of Formula (I) above may exist in unsolvated forms as well as solvated forms, such as, for example, hydrated forms. It is to be understood that the present invention encompasses all such solvated forms that activate TLR7.

It is also to be understood that certain compounds of the Formula (I) may exist in crystalline form and exhibit polymorphism. The present invention encompasses all such forms that activate TLR7.

The term “C₁₋₄alkyl” is intended to mean a saturated carbon chain of 1 to 4 carbon atoms in length which may be straight chained or branched. However references to individual alkyl groups such as “propyl” are specific for the straight chain version only and references to individual branched chain alkyl groups such as tert butyl are specific for the branched chain version only. For example, “C₁₋₄alkyl” includes, but is not limited to, methyl, ethyl, propyl, isopropyl, butyl, and tert-butyl. The term “C₂₋₄alkyl” and “C₁₋₃alkyl” are to be construed accordingly.

The term “C₁₋₃alkoxyC₂₋₄alkyl” is intended to mean a saturated carbon chain of 1 to 3 carbon atoms in length, which may be straight-chained or branched, linked via oxygen to another saturated carbon chain of 2 to 4 carbon atoms in length, which may be straight-chained or branched. For example, “C₁₋₃alkoxyC₂₋₄alkyl” includes, but is not limited to, methoxyethyl, methoxypropyl, ethoxypropyl, propoxyethyl and methoxypropyl.

The term “hydroxyC₂₋₄alkyl” is intended to mean a saturated carbon chain of 2 to 4 carbon atoms in length, which may be straight-chained or branched, wherein one of the hydrogen atoms has been replaced by a hydroxy group. For example “hydroxyC₂₋₄alkyl” includes, but is not limited to, hydroxyethyl, 2-hydroxypropyl, 3-hydroxypropyl, 1-hydroxyisopropyl and 4-hydroxybutyl.

The term “(R^{X})(R^{Y})N—C₂₋₄alkyl” is intended to mean a saturated carbon chain of 2 to 4 carbon atoms in length, which may be straight-chained or branched, wherein one of the hydrogen atoms has been replaced by a group “(R^{X})(R^{Y})N—”, wherein R^{X} and R^{Y} each independently represent hydrogen or C₁₋₃alkyl. The “C₁₋₃alkyl” is intended to mean a saturated carbon chain of 1 to 3 carbon atoms in length which may be straight chained or branched, including methyl, ethyl, propyl and isopropyl.

In one embodiment there is provided a compound of Formula (I) of Formula (IA):

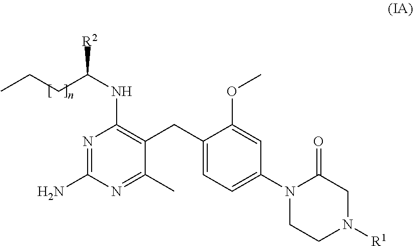

or a pharmaceutically acceptable salt thereof, wherein the values of R¹, R² and n may take any of the values defined herein for R¹, R² and n respectively.

The variable groups n, R′ and R² may also take any of the values as indicated below. Such values may be used together with any of the definitions, claims, aspects or embodiments defined herein to provide further embodiments or claims of the invention, and unless the context does not permit, any number of said variable group definitions may be used in any combination with each other to form further embodiments, aspects and claims. For example, the skilled person would understand that paragraphs (2), (4) and (9) could be combined to provided a compound of Formula (I), or a pharmaceutically acceptable salt thereof, wherein n represents 1, R¹ represents hydrogen, C₁₋₄alkyl, C₁₋₃alkoxyC₂₋₄alkyl, hydroxyC₂₋₄alkyl or (R^{X})(R^{Y})N—C₂₋₄alkyl, and R² represents hydrogen or 2-hydroxyethyl. Or, for example, the skilled person would understand that paragraphs (2), (7) and (8) could be combined to provided a compound of Formula (I), or a pharmaceutically acceptable salt thereof, wherein n represents 1, R¹ is methyl and R² is hydrogen, hydroxymethyl or 2-hydroxyethyl:
(1) n=1 or 2.
(2) n=1.
(3) n=2.
(4) R¹ is hydrogen, C₁₋₄alkyl, C₁₋₃alkoxyC₂₋₄alkyl, hydroxyC₂₋₄alkyl or (R^{X})(R^{Y})N—C₂₋₄alkyl.
(5) R¹ is hydrogen or C₁₋₄alkyl.
(6) R¹ is C₁₋₄alkyl.
(7) R¹ is methyl.
(8) R² is hydrogen, hydroxymethyl or 2-hydroxyethyl.
(9) R² is hydrogen or 2-hydroxyethyl.
(10) R² is hydrogen.
(11) R² is 2-hydroxymethyl.

Particular novel compounds of Formula (I) include, but are not limited to, the following compounds:
1-(4-{[2-Amino-4-(butylamino)-6-methylpyrimidin-5-yl]methyl}-3-methoxyphenyl)-4-methylpiperazin-2-one;
1-(4-{[2-Amino-4-methyl-6-(pentylamino) pyrimidin-5-yl]methyl}-3-methoxyphenyl)-4-methylpiperazin-2-one;
(S)-1-(4-{[2-Amino-4-(1-hydroxyhexan-3-ylamino)-6-methylpyrimidin-5-yl]methyl}-3-methoxyphenyl)-4-methylpiperazin-2-one;
(S)-1-(4-{[2-Amino-4-(1-hydroxypentan-2-ylamino)-6-methylpyrimidin-5-yl]methyl}-3-methoxyphenyl)-4-methylpiperazin-2-one;
(S)-1-(4-{[2-Amino-4-(1-hydroxyhexan-3-ylamino)-6-methylpyrimidin-5-yl]methyl}-3-methoxyphenyl)piperazin-2-one;
   and pharmaceutically acceptable salts thereof.

In one embodiment of the invention there is provided any Example described herein, or a pharmaceutically acceptable salt thereof.

A suitable pharmaceutically-acceptable salt of a compound of the Formula (I) is, for example, an acid-addition salt of a compound of the Formula (I), for example an acid-addition salt with an inorganic or organic acid such as hydrochloric, hydrobromic, sulphuric, trifluoroacetic, citric or maleic acid.

The compounds of the invention may be administered in the form of a pro-drug, that is a compound that is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in-vivo cleavable amide derivatives that may be formed at an amino group in a compound of the Formula (I).

Accordingly, the present invention includes those compounds of the Formula (I) as defined hereinbefore when made available by organic synthesis and when made available within the human or animal body by way of cleavage of a pro-drug thereof. Accordingly, the present invention includes those compounds of the Formula (I) that are produced by organic synthetic means and also such compounds that are produced in the human or animal body by way of metabolism of a precursor compound, that is a compound of the Formula (I) may be a synthetically-produced compound or a metabolically-produced compound.

A suitable pharmaceutically-acceptable pro-drug of a compound of the Formula (I) is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically-acceptable pro-drug of a compound of the Formula (I) that possesses an amino group is, for example, an in-vivo cleavable amide derivative thereof. Suitable pharmaceutically-acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl.

The in-vivo effects of a compound of the Formula (I) may be exerted in part by one or more metabolites that are formed within the human or animal body after administration of a compound of the Formula (I). As stated hereinbefore, the in-vivo effects of a compound of the Formula (I) may also be exerted by way of metabolism of a precursor compound (a pro-drug).

### Preparation of Compounds of Formula (I)

Compounds of Formula (I) may be prepared as described in the following reaction Schemes.

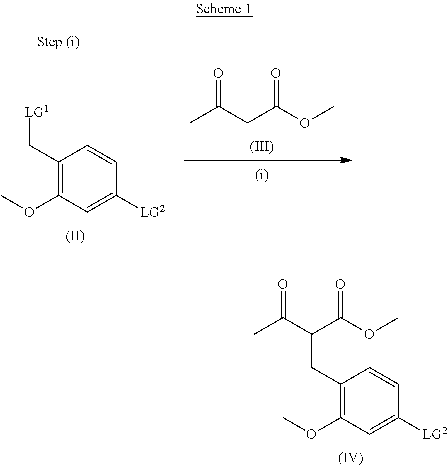

A compound of Formula (IV) may be prepared by reacting a compound of Formula (III) with a base, such as NaH, in a suitable solvent, such as THF or DMF, at a suitable temperature, for example 0° C. to r.t., followed by addition of a compound of Formula (II) wherein LG¹ and LG² each independently represent a suitable leaving group such as bromo, chloro or —OSO₂R³, wherein R³ represents C₁₋₃alkyl or optionally substituted aryl such as phenyl, 4-methylphenyl or 2,4,6-trimethylphenyl. The reaction mixture is then preferably heated, for example 50-100° C., optionally in the presence of an additive such as KI.

Alternatively the compound of Formula (IV) may be prepared by the following Scheme 1-2:

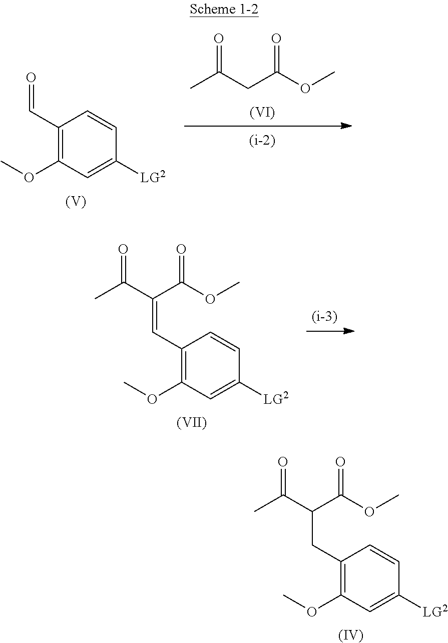

A compound of Formula (VII) may be prepared by reaction of a compound of Formula (V) with a compound of Formula (VI), as shown in Scheme 1-2. The reaction may be carried out in the presence of acetic acid and piperidine in a suitable solvent, such as toluene, and at a suitable temperature, for example 50-150° C. A compound of Formula (IV) can be prepared by reduction of a compound of Formula (VII) in the presence of a catalyst such as Pd/C under an atmosphere of H₂ (1-20 bar) in a suitable solvent, such as MeOH, and at a suitable temperature, for example 20-100° C.

A compound of Formula (IV) may also be synthesized by the following method, shown in Scheme 1-3.

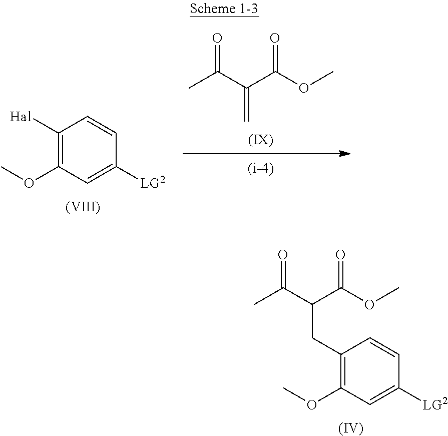

A compound of Formula (IV) may be prepared by a Heck reaction between a compound of Formula (VIII) and a compound of Formula (IX) as shown in Scheme 1-3 wherein Hal represents bromo or iodo. The reaction may be carried out using a palladium catalyst, such as palladium (II) acetate, bis(tri-o-tolylphosphine)palladium(II) dichloride or 1,1-bis(di-tert-butylphosphino)-ferrocenepalladium(II) chloride (Pd-118), a base such as NaHCO₃, triethylamine or dicyclohexylmethylamine, and tetrabutylammonium chloride or tetrabutylammonium bromide. The reaction is performed in a suitable solvent, such as THF, DMF or DMA, and at a suitable temperature, for example 50-150° C.

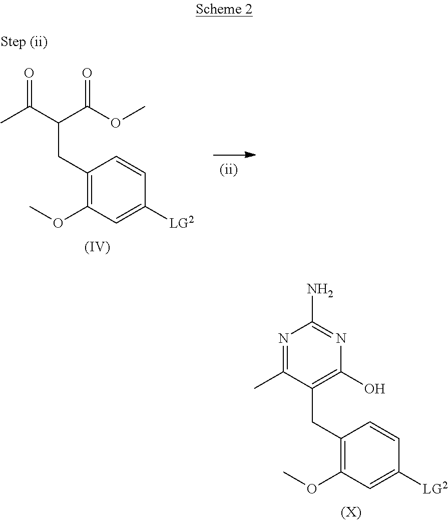

A compound of Formula (X) may be prepared by reacting a compound of Formula (IV) with guanidine or guanidine carbonate in a suitable solvent such as MeOH or EtOH, and at a suitable temperature, for example 50-150° C.

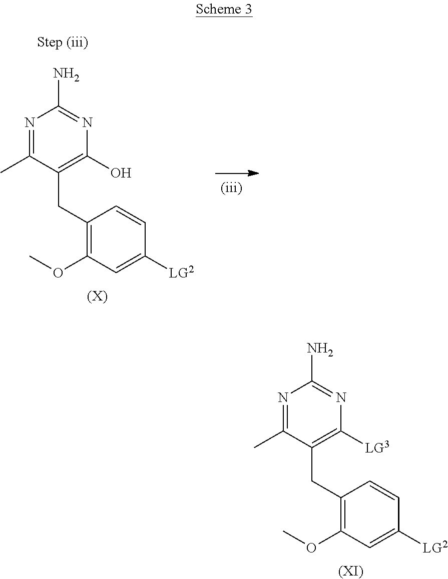

A compound of Formula (XI) may be prepared by reacting a compound of Formula (X) with POCl₃, at a suitable temperature, for example, 50-100° C., when LG³ represents chloro. Alternatively, a compound of Formula (XI) may also be prepared by reacting a compound of Formula (X) with a compound of formula R³SO₂Cl, wherein R³ represents C₁₋₃alkyl or optionally substituted aryl such as phenyl, 4-methylphenyl or 2,4,6-trimethylphenyl.

The reaction may be carried out in a suitable solvent, such as DCM or THF, in the presence of a suitable base, such as N,N,N′,N′-tetramethyl-1,3-propanediamine or triethylamine, at a suitable temperature, for example 0-50° C., when LG³ represents —OSO₂ R³.

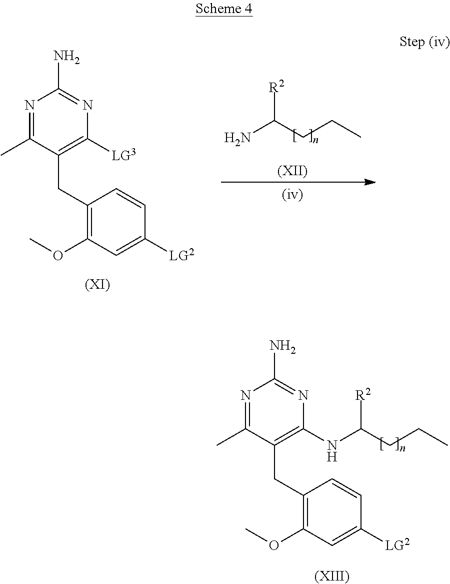

A compound of Formula (XIII) may be prepared by reacting a compound of Formula (XI) with an excess of an amine of Formula (XII) in a suitable solvent, such as butanol, propionitrile, butyl acetate or 1,4-dioxane, and at a suitable temperature, for example 50-150° C., optionally in the presence of an additive such as trifluoroacetic acid or DIPEA. Alternatively, the reaction may be performed in a microwave at a suitable temperature, for example 50-200° C.

In the Formula (XII) and/or (XIII), the hydroxyl group in R² may be protected with protective group such as benzyl or tert-butyldimethylsilyl, when R² represents hydroxymethyl or 2-hydroxyethyl. The hydroxyl group may be protected after the reaction of step (iv).

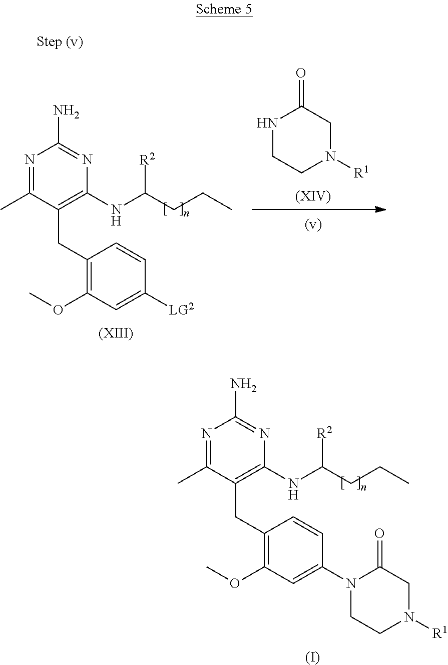

A compound of Formula (I) may be prepared by reaction a compound of Formula (XIII) with (XIV) in a suitable solvent, such as 1,4-dioxane, toluene or DMF, in the presence of a catalyst, such as CuI, additives such as N,N′-dimethyldiaminoethane or trans-1,2-cyclohexanediamine, and a suitable base, such as Cs₂CO₃ or K₂CO₃ and at a suitable temperature, for example 50-150° C.

When R¹ within a compound of Formula (I) is hydrogen, R¹ within a compound of Formula (XIV) may be protected by a protecting group such as tert-butoxycarbonyl group, which may be removed by an acid such as hydrochloric acid or TFA in a suitable solvent such as 1,4-dioxane or MeOH after reacting with a compound of Formula (XIII). When a hydroxyl group within R² is protected, the compound of Formula (I) may be prepared by additional steps of protecting the hydroxy group and removing the protective group, as in the following scheme 5-2 or Scheme 6:

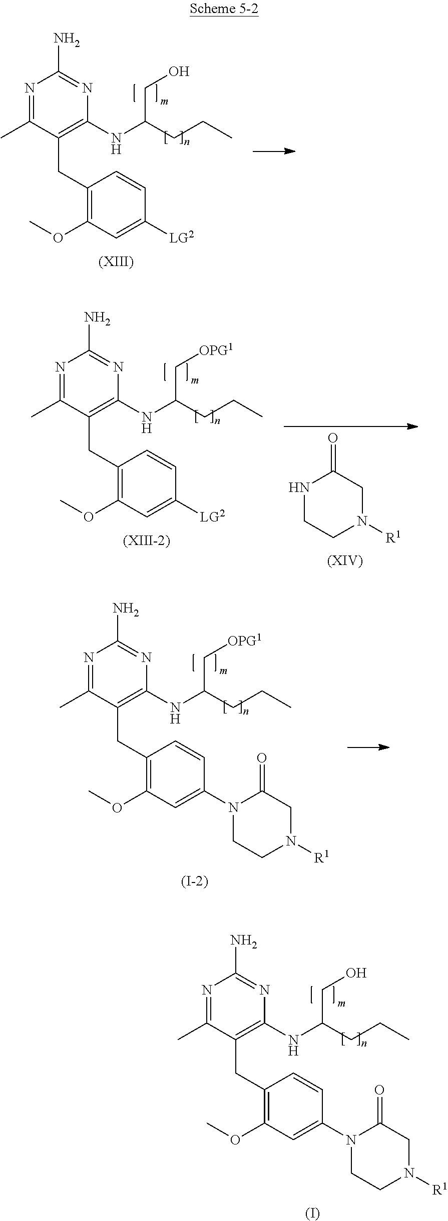

In Scheme 5-2, m represents 1 or 2, and PG¹ represents a protective group such as tert-butyldimethylsilyl (TBDMS) or benzyl. A compound of Formula (XIII-2) may be prepared by protecting the hydroxyl group of a compound of Formula (XIII) with protective group: PG¹ such as benzyl or TBDMS. A compound of Formula (I-2) may be prepared by the method described in step (v) of Scheme 5.

When the protective group represents benzyl ether, the deprotection reaction may be carried out with a catalyst such as Pd/C under an atmosphere of H₂ (1-20 bar) in a suitable solvent, such as MeOH, and at a suitable temperature, for example 20-100° C., optionally in the presence of an additive such as hydrochloric acid or acetic acid. When the protective group represents TBDMS, the protective group can be removed by the reaction with a fluoride compound such as tetra-n-butyl-ammonium fluoride (TBAF).

Alternatively, a compound of Formula (XVIII) in the Scheme 6 may be used instead of a compound of Formula (MI) in the reaction process of Scheme 4. A compound of Formula (XVIII), wherein a hydroxyl group within R² is protected, may be prepared by the Scheme 6:

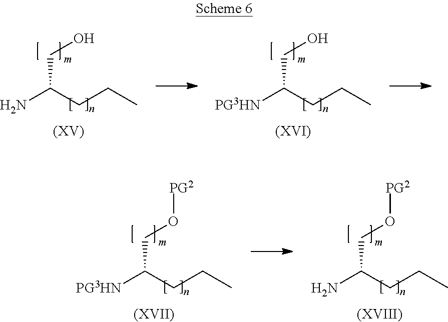

A compound of Formula (XVI) may be prepared by protecting the amino group of a compound of Formula (XV), wherein m represents 1 or 2, with protective group: PG³ such as tert-butoxycarbonyl by a reaction of a compound of Formula (XV) with di-tert-butyl dicarbonate in the presence of a base such as NaOH in a suitable solvent, such as methanol and water at a suitable temperature, for example 0-50° C. A compound of Formula (XVII) may be prepared by protecting the hydroxyl group of a compound of Formula (XVI) with protective group: PG² such as benzyl by a reaction of a compound of Formula (XVI) with benzyl bromide or chloride in the presence of a base such as NaH or DIPEA in a suitable solvent, such as DMF at a suitable temperature, for example 0-50° C. A compound of Formula (XVIII), wherein a hydroxyl group within R² of a compound of Formula (XII) is protected by PG², may be prepared by de-protecting the amino group with an acid such as hydrochloric acid or TFA in a suitable solvent, such as 1,4-dioxane or MeOH at a suitable temperature, for example 0-50° C., when PG³ is tert-butoxycarbonyl.

The conditions used for each reaction step is described in the examples in this specification.

Alternatively, a compound of Formula (I) may be prepared by the method shown in Scheme 7:

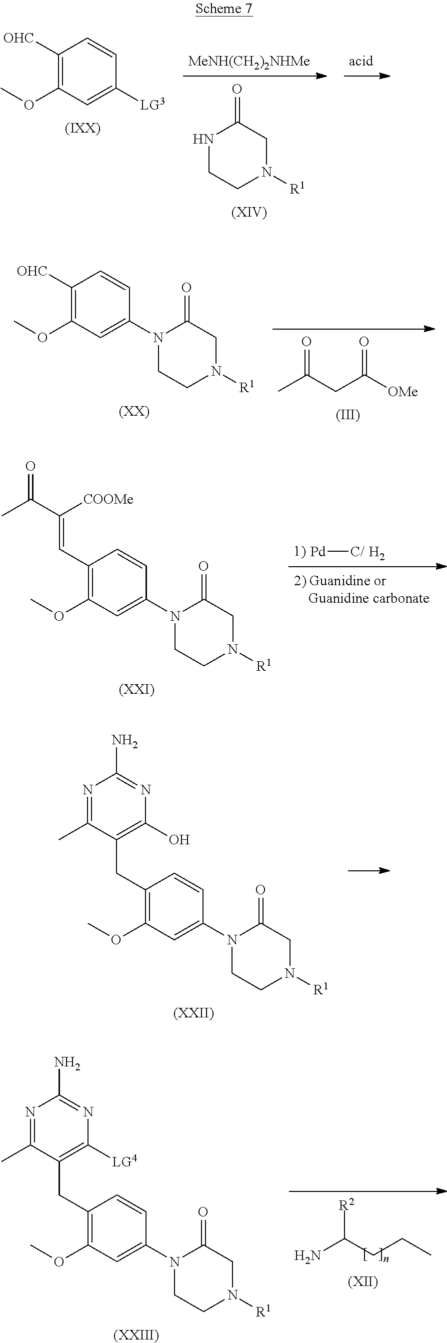

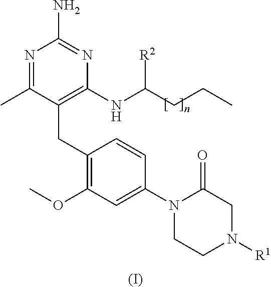

wherein LG³ and LG⁴ each independently represent a suitable leaving group such as chloro, bromo or —OSO₂R³, and wherein R³ represents C₁₋₃alkyl or an optionally substituted aryl such as phenyl, 4-methylphenyl or 2,4,6-trimethylphenyl. A compound of Formula (XX) may be prepared by reacting a compound of Formula (IXX) with a compound Formula (XIV) in a suitable solvent, such as 1,4-dioxane, toluene or DMF in the presence of a catalyst, such as CuI, additives such as N,N′-dimethyldiaminoethane or trans-1,2-cyclohexanediamine, and a base, such as Cs₂CO₃ or K₂CO₃ and at a suitable temperature, for example 50-150° C., and followed by reacting with an acid such as aqueous hydrochloride or silica gel. A compound of Formula (XXI) may be prepared by reacting a compound of Formula (XX) with a compound of Formula (III) in the presence of acetic acid and piperidine in a suitable solvent such as toluene, and at a suitable temperature, for example 50-150° C. A compound of Formula (XXII) may be prepared by reduction of a compound of Formula (XXI) in the presence of catalyst such as Pd/C under an atmosphere of H₂ (1-20 bar) in a suitable solvent, such as MeOH and at a suitable temperature, for example 20-100° C., and followed by reacting with a guanidine or a guanidine carbonate in a suitable solvent such as MeOH or EtOH, and at a suitable temperature, for example 50-100° C. A compound of Formula (XXIII) may be prepared by reacting a compound of Formula (XXII) with POCl₃, at a suitable temperature, for example, 50-150° C., when LG⁴ represents chloro. Alternatively, a compound of Formula (XXIII) may be prepared by reacting a compound of Formula (XXII) with a compound of formula R³SO₂Cl, wherein R³ represents C₁₋₃ alkyl or optionally substituted aryl such as phenyl, 4-methylphenyl or 2,4,6-trimethylphenyl. The reaction may be carried out in a suitable solvent, such as DCM or THF, in the presence of a suitable base, such as N,N,N′,N′-tetramethyl-1,3-propanediamine or triethylamine, at a suitable temperature, for example 0-50° C., when LG⁴ represents —OSO₂R³. A compound of Formula (I) may be prepared by reacting with an excess of an amine of Formula (XII) in a suitable solvent such as butanol, propionitrile, butyl acetate or 1,4-dioxane, and at a suitable temperature, for example 50-150° C., optionally in the presence of an additive such as trifluoroacetic acid or DIPEA. Alternatively, the reaction may be performed in a microwave at a suitable temperature, for example 50-200° C.

The compounds described herein in the Schemes and associated text, and later in the detailed experimental section, are useful intermediates for the preparation of the compounds of Formula (I) and may be isolated as a free base or as a salt. Therefore, in further aspects and embodiments of the invention there is provided an intermediate described herein, or a salt thereof, wherein any of the variable groups described for said intermediate may take any of the values described herein in connection with that group.

The intermediates having an R² group wherein R² is other than hydrogen have a chiral centre at the carbon atom to which the R² group is attached. In further embodiments there is provided each of such intermediates, or a salt thereof, wherein the intermediate has the (S)-stereochemical configuration.

Compounds of Formula (II), (III), (V), (VI), (VIII), (IX), (XII), (XIV) and (XV) are known compounds or can be prepared from known compounds by conventional methods, or their syntheses are described herein.

It will be appreciated by those skilled in the art that in the processes of the present invention certain functional groups such as hydroxyl or amino groups in the reagents may need to be protected by protecting groups. Thus, the preparation of the compounds of Formula (I) may involve, at an appropriate stage, the removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3rd edition, T. W. Greene and P. G. M. Wuts, Wiley-Interscience (1999).

According to a further aspect of the invention there is provided a pharmaceutical composition which comprises a compound of the Formula (I), or a pharmaceutically acceptable salt thereof, as defined hereinbefore in association with a pharmaceutically-acceptable diluent or carrier. The pharmaceutical composition may be used in the treatment of cancer. The composition may be in a form suitable for oral administration, for example as a tablet or capsule; for parenteral injection (including intravenous, subcutaneous, intramuscular, intravascular or infusion) as a sterile solution, suspension or emulsion; for topical administration as an ointment or cream; or for rectal administration as a suppository.

The compound of Formula (I), or a pharmaceutically acceptable salt thereof, could also be administered as an air spray for inhalation. The air spray (e.g., spray, aerosol, dry powder preparation, etc.) could be optionally formulated as an aqueous solution or suspension, or as an aerosol delivered from a pressurized pack such as a pressurised metered dose inhaler by using, for example, a liquefied propellant. A dry powder preparation may also be used. An aerosol appropriate for inhalation may be either a suspension or solution, and would typically contain the compound of Formula (I), or a pharmaceutically acceptable salt thereof, and any appropriate propellants such as a fluorocarbon or hydrogen-containing chlorofluorocarbon or a mixture thereof. Specifically, it may contain hydrofluoroalkane, particularly 1,1,1,2-tetrafluoroethane, heptafluoroalkane (HFA) such as 1,1,1,2,3,3,3-heptafluoro-n-propane, or a mixture thereof. An aerosol may optionally contain an additional preparation excipient well-known to those skilled in the art such as surfactant (e.g., oleic acid or lecithin) and cosolvent (e.g., ethanol), etc. Specifically, an aerosol preparation could be delivered using the inhaler known as “Turbuhaler™”.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compound of Formula (I) will normally be administered to a warm blooded animal at a unit dose within the range 5-5000 mg/m² body area of the animal, i.e. approximately 0.1-100 mg/kg, and this normally provides a therapeutically effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

In the context of the present specification, the term “therapy” also includes “prophylaxis” unless there are specific indications to the contrary. The terms “therapeutic” and “therapeutically” should be construed accordingly.

As used herein, the term “treatment” is intended to have its normal everyday meaning of dealing with a disease in order to entirely or partially relieve one, some or all of its symptoms, or to correct or compensate for the underlying pathology.

As used herein, the term “prophylaxis” is intended to have its normal everyday meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

The compounds defined in the present invention are effective activators of TLR7 in vitro. Accordingly, the compounds of the present invention are expected to be potentially useful agents in the treatment of diseases or medical conditions mediated alone or in part by TLR7. For example, the following diseases and conditions listed in paragraphs 1 to 8 below may be treatable with compounds of the present invention.

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
3. eyes: blepharitis; conjunctivitis, including perennial and vernal allergic conjunctivitis; iritis; anterior and posterior uveitis; choroiditis; autoimmune, degenerative or inflammatory disorders affecting the retina; ophthalmitis including sympathetic ophthalmitis; sarcoidosis; infections including viral, fungal, and bacterial;
4. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
5. allograft rejection: acute and chronic following, for example, transplantation of kidney, heart, liver, lung, bone marrow, skin or cornea or following blood transfusion; or chronic graft versus host disease;
6. other auto-immune and allergic disorders including rheumatoid arthritis, irritable bowel syndrome, systemic lupus erythematosus, multiple sclerosis, Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome and Sazary syndrome;
7. oncology: treatment of common cancers including bladder, head and neck, prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
8. infectious diseases: virus diseases such as genital warts, common warts, plantar warts, hepatitis B, hepatitis C, herpes simplex virus, molluscum contagiosum, variola, human immunodeficiency virus (HIV), human papilloma virus (HPV), cytomegalovirus (CMV), varicella zoster virus (VZV), rhinovirus, adenovirus, coronavirus, influenza, para-influenza; bacterial diseases such as tuberculosis and mycobacterium avium, leprosy; other infectious diseases, such as fungal diseases, chlamydia, candida, aspergillus, cryptococcal meningitis, pneumocystis carnii, cryptosporidiosis, histoplasmosis, toxoplasmosis, trypanosome infection and leishmaniasis.

It is envisaged that for the methods of treatment mentioned herein, the compound of Formula (I) will be administered to a mammal, more particularly a human being. Similarly, for the uses of a compound of Formula (I) for the treatment of diseases or medical conditions mentioned herein, it is envisaged that the compound of Formula (I) will be administered to a mammal, more particularly a human being.

According to a another aspect of the invention, there is therefore provided a compound of Formula (I) as defined hereinbefore, or a pharmaceutically acceptable salt thereof, for use as a medicament.

According to a further aspect of the invention, there is provided a compound of Formula (I) as defined hereinbefore, or a pharmaceutically acceptable salt thereof for use in the treatment of a disease mediated through TLR7. In one embodiment of the invention, said disease mediated through TLR7 is cancer. In a further embodiment of the invention, said cancer is selected from bladder cancer, head and neck cancer, prostate cancer, breast cancer, lung cancer, uterus cancer, pancreatic cancer, liver cancer, renal cancer, ovarian cancer, colon cancer, stomach cancer, skin cancer, cerebral tumor, malignant myeloma and lymphoproliferative tumors. In one embodiment of the invention, said disease mediated through TLR7 is asthma, COPD, allergic rhinitis, allergic conjunctivitis, atopic dermatitis, hepatitis B, hepatitis C, HIV, HPV, bacterial infections or dermatosis.

According to a further aspect of the invention, there is provided the use of a compound of Formula (I) as defined hereinbefore, or a pharmaceutically acceptable salt thereof for the preparation of a medicament for the treatment of a disease mediated through TLR7. In one embodiment of the invention, said disease mediated through TLR7 is cancer. In a further embodiment of the invention, said cancer is selected from bladder cancer, head and neck cancer, prostate cancer, breast cancer, lung cancer, uterus cancer, pancreatic cancer, liver cancer, renal cancer, ovarian cancer, colon cancer, stomach cancer, skin cancer, cerebral tumor, malignant myeloma and lymphoproliferative tumors. In one embodiment of the invention, said disease mediated through TLR7 is asthma, COPD, allergic rhinitis, allergic conjunctivitis, atopic dermatitis, hepatitis B, hepatitis C, HIV, HPV, bacterial infections or dermatosis.

According to a further aspect of the invention, there is provided the use of a compound of Formula (I) as defined hereinbefore, or a pharmaceutically acceptable salt thereof, for the preparation of a medicament for the treatment of cancer. In one embodiment of the invention, said cancer is selected from bladder cancer, head and neck cancer, prostate cancer, breast cancer, lung cancer, uterus cancer, pancreatic cancer, liver cancer, renal cancer, ovarian cancer, colon cancer, stomach cancer, skin cancer, cerebral tumor, malignant myeloma and lymphoproliferative tumors.

According to a further aspect of the invention, there is provided the use of a compound of Formula (I) as defined hereinbefore, or a pharmaceutically acceptable salt thereof, for the preparation of a medicament for the treatment of asthma, COPD, allergic rhinitis, allergic conjunctivitis, atopic dermatitis, hepatitis B, hepatitis C, HIV, HPV, bacterial infections or dermatosis.

In one aspect of the invention there is provided the use of a compound of Formula (I) or a pharmaceutically acceptable salt thereof for use in the treatment of cancer.

According to a further aspect of the invention, there is provided a method of using a compound of Formula (I) as defined hereinbefore, or a pharmaceutically acceptable salt thereof, for the treatment of cancer. Accordingly there is therefore provided a method of treating cancer in a warm-blooded animal, such as man, in need of such treatment, which comprises administering to said animal an effective amount of the compound of Formula (I), or a pharmaceutically acceptable salt thereof, as defined herein. In one embodiment of the invention, said cancer is selected from bladder cancer, head and neck cancer, prostate cancer, breast cancer, lung cancer, uterus cancer, pancreatic cancer, liver cancer, renal cancer, ovarian cancer, colon cancer, stomach cancer, skin cancer, cerebral tumor, malignant myeloma and lymphoproliferative tumors.

According to a further aspect of the invention, there is provided a method of using a compound of Formula (I) as defined hereinbefore, or a pharmaceutically acceptable salt thereof, for the treatment of asthma, COPD, allergic rhinitis, allergic conjunctivitis, atopic dermatitis, hepatitis B, hepatitis C, HIV, HPV, bacterial infections or dermatosis.

According to a further aspect of the invention, there is provided a method of treating a human suffering from a disease in which activation of TLR7 is beneficial, comprising the steps of administering to a person in need thereof of a therapeutically effective amount of a compound of Formula (I) as defined hereinbefore, or a pharmaceutically acceptable salt thereof. In one embodiment of the invention, the disease in which activation of TLR7 is beneficial is cancer. In a further embodiment of the invention, said cancer is selected from bladder cancer, head and neck cancer, prostate cancer, breast cancer, lung cancer, uterus cancer, pancreatic cancer, liver cancer, renal cancer, ovarian cancer, colon cancer, stomach cancer, skin cancer, cerebral tumor, malignant myeloma and lymphoproliferative tumors. In one embodiment of the invention, the disease in which activation of TLR7 is beneficial is asthma, COPD, allergic rhinitis, allergic conjunctivitis, atopic dermatitis, hepatitis B, hepatitis C, HIV, HPV, bacterial infections or dermatosis.

In any aspect or embodiment described herein the cancer may be bladder cancer.

In any aspect or embodiment described herein the cancer may be head and neck cancer.

In any aspect or embodiment described herein the cancer may be prostate cancer.

In any aspect or embodiment described herein the cancer may be breast cancer.

In any aspect or embodiment described herein the cancer may be lung cancer.

In any aspect or embodiment described herein the cancer may be uterus cancer.

In any aspect or embodiment described herein the cancer may be pancreatic cancer.

In any aspect or embodiment described herein the cancer may be liver cancer.

In any aspect or embodiment described herein the cancer may be renal cancer.

In any aspect or embodiment described herein the cancer may be ovarian cancer.

In any aspect or embodiment described herein the cancer may be colon cancer.

In any aspect or embodiment described herein the cancer may be stomach cancer.

In any aspect or embodiment described herein the cancer may be skin cancer.

In any aspect or embodiment described herein the cancer may be cerebral tumor.

In any aspect or embodiment described herein the cancer may be malignant myeloma cancer.

In any aspect or embodiment described herein the cancer may be lymphoproliferative tumors.

The anti-cancer treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis platin, miriplatin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, amurubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI 774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459), cyclin dependent kinase inhibitors such as CDK2 and/or CDK4 inhibitors and BRAF inhibitors such as Vemurafenib;
(v) antiangiogenic agents such as those which inhibit the effects of vascular endothelial growth factor, [for example the anti vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(x) immunotherapy approaches, including for example ex vivo and in vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies, approaches to decrease the function of immune suppressive cells such as regulatory T cells, myeloid-derived suppressor cells or IDO (indoleamine 2,3,-deoxygenase)-expressing dendritic cells, and approaches using cancer vaccines consisting of proteins or peptides derived from tumour-associated antigens such as NY-ESO-1, MAGE-3, WT1 or Her2/neu.

According to this aspect of the invention there is provided a pharmaceutical product comprising a compound of the formula (I) as defined hereinbefore and an additional anti tumour substance as defined hereinbefore for the conjoint treatment of cancer.

According to this aspect of the invention there is provided a pharmaceutical product comprising a compound of the Formula (I), or a pharmaceutically acceptable salt thereof, as defined hereinbefore, and an additional anti tumour substance for the conjoint treatment of cancer.

According to this aspect of the invention there is provided a combination suitable for use in the treatment of cancer comprising a compound of Formula (I) as defined hereinbefore,

or a pharmaceutically acceptable salt thereof, and any one of the anti tumour agents listed under (i)-(ix) above.

Therefore in a further aspect of the invention there is provided a compound of Formula (I), or a pharmaceutically acceptable salt thereof, in combination with an anti-tumour agent selected from one listed under (i)-(ix) herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of Formula (I), or a pharmaceutically acceptable salt thereof, in combination with an anti-tumour agent selected from one listed under (i)-(ix) herein above, in association with a pharmaceutically acceptable diluent or carrier.

According to a further aspect of the invention there is provided a pharmaceutical composition which comprises a compound of Formula (I), or a pharmaceutically acceptable salt thereof, in combination with an anti-tumour agent selected from one listed under (i)-(ix) herein above, in association with a pharmaceutically acceptable diluent or carrier for use in the treatment of cancer.

According to another feature of the invention there is provided the use of a compound of the Formula (I), or a pharmaceutically acceptable salt thereof, in combination with an anti-tumour agent selected from one listed under (i)-(ix) herein above, in the manufacture of a medicament for use in cancer in a warm blooded animal, such as man.

According to another feature of the invention there is provided a compound of the Formula (I), or a pharmaceutically acceptable salt thereof, in combination with an anti-tumour agent selected from one listed under (i)-(ix) herein above for use in the treatment of cancer in a warm blooded animal, such as man.

Therefore in an additional feature of the invention, there is provided a method of treating cancer in a warm blooded animal, such as man, in need of such treatment which comprises administering to said animal an effective amount of a compound of Formula (I), or a pharmaceutically acceptable salt thereof, in combination with an anti-tumour agent selected from one listed under (i)-(ix) herein above.

According to a further aspect of the present invention there is provided a kit comprising a compound of Formula (I), or a pharmaceutically acceptable salt thereof, in combination with an anti-tumour agent selected from one listed under (i)-(ix) herein above.

According to a further aspect of the present invention there is provided a kit comprising:

a) a compound of Formula (I), or a pharmaceutically acceptable salt thereof, in a first unit dosage form;
b) an anti-tumour agent selected from one listed under (i)-(ix) herein above; in a second unit dosage form; and
c) container means for containing said first and second dosage forms.

According to a further aspect of the present invention there is provided a kit comprising a compound of Formula (I), or a pharmaceutically acceptable salt thereof, in combination with an additional anti-tumour agent.

According to a further aspect of the present invention there is provided a kit comprising:

a) a compound of Formula (I), or a pharmaceutically acceptable salt thereof, in a first unit dosage form;
b) a second anti-tumour agent in a second unit dosage form; and
c) container means for containing said first and second dosage forms.

In one aspect of the invention the compounds of Formula (I) may be useful as vaccine adjuvants.

As a further aspect of the invention there is provided a compound of formula (I), or a pharmaceutically acceptable salt thereof, as defined herein, for use as a vaccine adjuvant.

As a further aspect of the invention there is provided the use of a compound of formula (I), or a pharmaceutically acceptable salt thereof, as defined herein, as a vaccine adjuvant, in the manufacture of a vaccine for the treatment of a disease or condition.

The invention still further provides a method of treating, or reducing the risk of, a disease or condition, which method comprises administering to a patient in need thereof a therapeutically effective amount of a vaccine and a compound of formula (I), or a pharmaceutically acceptable salt thereof, as defined herein.

The invention still further provides a method of increasing the response to a vaccine in a patient, which method comprises administering to a patient in need thereof a therapeutically effective amount of a vaccine and a compound of formula (I), or a pharmaceutically acceptable salt thereof, as defined herein.

### EXAMPLES

The invention will now be illustrated with the following Examples in which generally, unless stated otherwise, all starting materials are commercially available. “r.t.” means “room temperature”, i.e. in the range 17 to 28° C., typically 20° C. Electrospray ionization mass spectrometry (ESI mass) spectra were recorded using Waters Micromass ZQ™ mass spectrometer, Waters Waters 2790 Alliance™ separation module and Imtakt Cadenza CD-C18™ columneluting with a gradient of A (MeCN), B (H₂O) and C (2% formic acid/98% MeCN). 0.0-0.1 min: A 95%, B 2% and C 3%. 0.1-3.1 min: linear gradient from A 95%, B 2% and C 3% to A 1%, B 96% and C 3%. 3.1-3.5 min: A 1%, B 96% and C 3%.

Proton nuclear magnetic resonance (¹H NMR) spectra were recorded at 300-500 MHz using deuterated chloroform, unless otherwise stated. The following abbreviations are used for NMR data: s=singlet, d=doublet, t=triplet, q=quartet, m=multiplet, dd=doublet of doublets, tt=triplet of triplets, br=broad, quintet=qn.

The following abbreviations may be used in the scientific parts of this specification: EtOAc=ethyl acetate; DCM=dichloromethane; NMP=N-methylpyrrolidinone; DMF=N,N-dimethylformamide; DMSO=dimethylsulfoxide; THF=tetrahydrofuran; MeOH=methanol; EtOH=ethanol; MeCN=acetonitrile; Pd/C=palladium on carbon; DMAP=4-dimethylaminopyridine; Mes=mesitylenyl (2,4,6-trimethylphenyl); sat.=saturated; aq.=aqueous; DMA=N,N-dimethylacetamide; conc.=concentrated; h=hours; min(s)=mins.; M=molar; MS=mass spectrometry; ESI=electron spray ionisation method; HPLC=high performance liquid chromatography; RPHPLC=reverse-phase high performance liquid chromatography; DIPEA=diisopropylethylamine.

### Example 1

### 1-(4-{[2-Amino-4-(butylamino)-6-methylpyrimidin-5-yl]methyl}-3-methoxyphenyl)-4-methylpiperazin-2-one

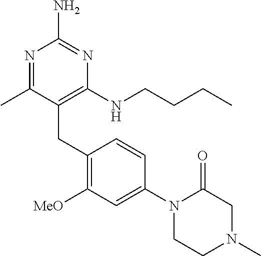

The title compound may be prepared by the steps described below:

### (i) Methyl 2-(4-bromo-2-methoxybenzyl)-3-oxobutanoate

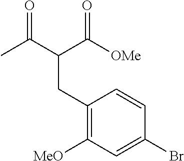 [SMILES: COC(=O)C(Cc1ccc(Br)cc1OC)C(C)=O]

To a stirred solution of 4-bromo-2-methoxybenzyl alcohol (11.0 g, 50.7 mmol) in CHCl₃ (100 mL) was added SOCl₂ (14.5 mL, 200 mmol) dropwise at 4° C. After the addition, the mixture was allowed to warm to r.t. and was stirred for 6 h. The solvent was evaporated and water was added. The resulting mixture was extracted with EtOAc, and the combined organic solutions were washed with sat. aq. NaHCO₃, brine, and then dried (Na₂SO₄). After removal of the solvent in vacuo, the resulting crude benzyl chloride derivative was used for the next step without further purification.

To a stirred suspension of NaH (2.55 g, 58.4 mmol, 55% in mineral oil) in DMF (120 mL) at r.t. was added methyl acetylacetate (6.21 g, 53.6 mmol). After stirring for 30 min, KI (8.47 g, 51.0 mmol) and the above benzyl chloride derivative was added. The resulting mixture was heated to 50° C. and stirred for 4 h. After cooling to r.t., sat. aq. NH₄Cl was added and the resulting mixture was extracted with EtOAc. The combined organic solutions were washed with brine, and then dried (Na₂SO₄). After removal of the solvent in vacuo, the crude residue was purified by silica gel column chromatography to give the subtitle compound as a pale yellow oil (13.0 g, 41.5 mmol, 82%); LC-MS: m/z=315 [MH⁺] (T=2.26 min).

### (ii) 2-Amino-5-(4-bromo-2-methoxybenzyl)-6-methylpyrimidin-4-ol

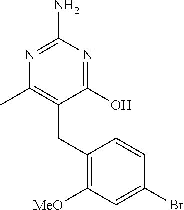 [SMILES: COc1cc(Br)ccc1Cc1c(C)nc(N)nc1O]

To a solution of the product from step (i) (13.0 g, 41.4 mmol) in MeOH (100 mL) was added guanidine carbonate (16.2 g, 90.0 mmol). The mixture was stirred for 12 h at reflux temperature. After cooling to r.t., some solvent was removed by evaporation to give a solution with half of the original volume. The resulting precipitate was collected by filtration and rinsed with water and MeOH to give the subtitle compound as a white solid (4.10 g, 12.8 mmol, 31%); ¹H NMR: (d⁶-DMSO) 7.10 (1H, d), 7.00 (1H, dd), 6.78 (1H, d), 6.40 (2H, br-s), 3.83 (s, 3H), 3.48 (s, 2H), 1.92 (s, 3H); LC-MS: m/z=324 [MH⁺] (T=1.75 min).

### (iii) 2-Amino-5-(4-bromo-2-methoxybenzyl)-6-methylpyrimidin-4-yl-2,4,6-trimethylbenzenesulfonate

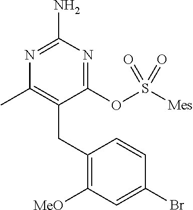

To a stirred solution of the product from step (ii) (1.50 g, 4.64 mmol) in THF (20 mL) at r.t. was added N,N,N′,N′-tetramethyl-1,3-propanediamine (1.17 mL, 7.00 mmol) and 2-mesitylenesulfonyl chloride (1.53 g, 7.00 mmol). After stirring for 12 h, water was added and the resulting mixture was extracted with EtOAc. The combined organic solutions were washed with brine, and then dried (Na₂SO₄). After removal of the solvent in vacuo, the crude residue was washed with diethyl ether/hexane (1/4) to give subtitle compound as a white solid (2.00 g, 3.94 mmol, 85%); ¹H NMR: 6.95-6.91 (4H, m), 6.69 (1H, d), 4.94 (2H, br s), 3.81 (3H, s), 3.76 (2H, s), 2.56 (6H, s), 2.32 (3H, s), 2.29 (3H, s); LC-MS: m/z=506 [MH⁺] (T=2.05).

### (iv) 5-(4-Bromo-2-methoxybenzyl)-N⁴-butyl-6-methylpyrimidine-2,4-diamine

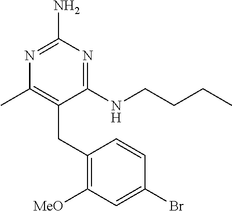 [SMILES: CCCCNc1nc(N)nc(C)c1Cc1ccc(Br)cc1OC]

To a solution of the product from step (iii) (0.250 g, 0.494 mmol) in propionitrile was added butylamine (0.240 mL, 2.47 mmol) and trifluoroacetic acid (0.0740 mL, 1.00 mmol). The mixture was heated to 110° C. and stirred for 7 h. After cooling to r.t., sat aq. NaHCO₃ was added and the resulting mixture was extracted with EtOAc. The combined organic solutions were washed with brine, and then dried (Na₂SO₄). After removal of the solvent in vacuo, the crude residue was purified by silica gel column chromatography to give the subtitle compound as a white solid (0.144 g, 0.381 mmol, 77%); LC-MS: m/z=393 [MH⁺] (T=2.05).

### (v) 1-(4-{[2-Amino-4-(butylamino)-6-methylpyrimidin-5-yl]methyl}-3-methoxyphenyl)-4-methylpiperazin-2-one

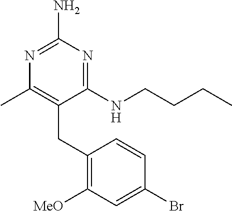 [SMILES: CCCCNc1nc(N)nc(C)c1Cc1ccc(Br)cc1OC]

To a solution of the product from step (iv) (0.144 g, 0.380 mmol) in 1,4-dioxane (2 mL) was added CuI (72.0 mg, 0.379 mmol), N,N′-dimethyldiaminoethane (0.0820 mL, 0.763 mmol), 4-methylpiperazin-2-one (87.0 mg, 0.760 mmol), and Cs₂CO₃ (247 mg, 0.760 mmol). The mixture was heated to 100° C. and stirred for 10 h. After cooling, water was added and the resulting mixture was extracted with EtOAc. The combined organic solutions were washed with brine, and then dried (Na₂SO₄). After removal of the solvent, in vacuo, the crude residue was purified by silica gel column chromatography to give the title compound as a pale yellow solid (120 mg, 0.291 mmol, 77%); ¹H NMR: 6.90 (1H, d), 6.86 (1H, s,), 6.72 (1H, d), 4.76 (3H, br s), 3.87 (3H, s), 3.68-3.65 (2H, m), 3.63 (2H, s,), 3.32-3.27 (2H, m), 3.26 (2H, s), 2.78-2.75 (2H, m), 2.39 (3H, s), 2.27 (3H, s), 1.45-1.37 (2H, m), 1.26-1.19 (2H, m), 0.85 (3H, t); LC-MS: m/z=413 [MH⁺] (T=1.48 min).

### Example 2

### 1-(4-{[2-Amino-4-methyl-6-(pentylamino)pyrimidin-5-yl]methyl}-3-methoxyphenyl)-4-methylpiperazin-2-one

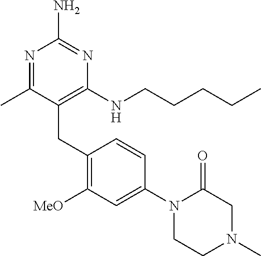 [SMILES: CCCCCNc1nc(N)nc(C)c1Cc1ccc(N2CCN(C)CC2=O)cc1OC]

The title compound may be prepared by the following steps:

### (i) 5-(4-Bromo-2-methoxybenzyl)-6-methyl-N⁴-pentylpyrimidine-2,4-diamine

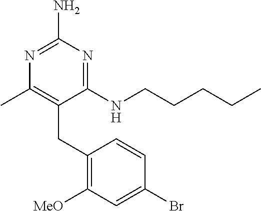 [SMILES: CCCCCNc1nc(N)nc(C)c1Cc1ccc(Br)cc1OC]

The subtitle compound was prepared using the product from Example 1 step (iii) (1.51 g, 3.00 mmol) and the method of Example 1 step (iv), in which pentylamine (1.05 mL, 9.04 mmol) was used instead of butylamine to give the subtitle compound as a pale yellow solid (1.00 g, 2.54 mmol, 85%); LC-MS: m/z=393 [MH⁺] (T=2.05).

### (ii) 1-(4-{[2-Amino-4-methyl-6-(pentylamino)pyrimidin-5-yl]methyl}-3-methoxyphenyl)-4-methylpiperazin-2-one

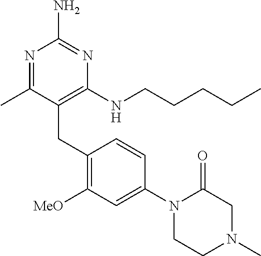

The title compound was prepared by the method of Example 1 step (v) using the product from step (i) (60.0 mg, 0.153 mmol) to give the title compound as a colourless oil (23.7 mg, 0.0556 mmol, 36%); ¹H NMR: 6.92 (1H, d), 6.88 (1H, d), 6.73 (1H, dd), 4.99 (3H, br s), 3.88 (3H, s), 3.67-3.63 (2H, m), 3.64 (2H, s,), 3.33-3.27 (2H, m), 3.26 (2H, s), 2.79-2.76 (2H, m), 2.39 (3H, s), 2.30 (3H, s), 1.48-1.41 (2H, m), 1.29-1.17 (4H, m), 0.85 (3H, t); LC-MS: m/z=427 [MH⁺] (T=1.66).

### Example 3

### (S)-1-(4-{[2-Amino-4-(1-hydroxyhexan-3-ylamino)-6-methylpyrimidin-5-yl]methyl}-3-methoxyphenyl)-4-methylpiperazin-2-one

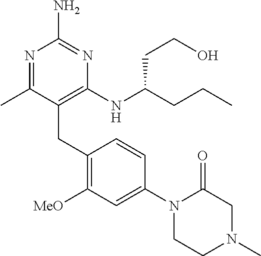

The title compound may be prepared by the following steps:

### (i) (S)-3-[2-Amino-5-(4-bromo-2-methoxybenzyl)-6-methylpyrimidin-4-ylamino]hexan-1-ol

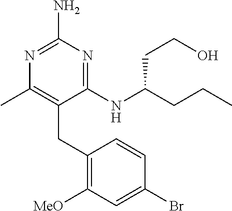 [SMILES: CCC[C@@H](CCO)Nc1nc(N)nc(C)c1Cc1ccc(Br)cc1OC]

The subtitle compound was prepared using the product from Example 1 step (iii) (0.770 g, 1.52 mmol) and the method of Example 1 step (iv), in which (S)-3-aminohexan-1-ol (0.890 g, 7.60 mmol) was used instead of butylamine to give the subtitle compound as a white amorphous solid (0.54 g, 1.28 mmol, 84%); LC-MS: m/z=423 [MH⁺] (T=1.90).

### (ii) (S)-5-(4-Bromo-2-methoxybenzyl)-N⁴-[1-(tert-butyldimethylsilyloxy)hexan-3-yl]-6-methylpyrimidine-2,4-diamine

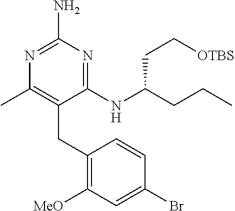 [SMILES: CCC[C@@H](CCO[Si](C)(C)C(C)(C)C)Nc1nc(N)nc(C)c1Cc1ccc(Br)cc1OC]

To a solution of the product from step (i) (0.430 g, 1.00 mmol) in DMF (5 mL) was added triethylamine (0.690 mL, 5.00 mmol) and tert-butyldimethylsilyl chloride (0.375 g, 2.50 mmol). After stirring for 10 h at r.t., sat. aq. NaHCO₃ was added, and the resulting mixture was extracted with EtOAc. The combined organic solutions were washed with brine, and then dried (Na₂SO₄). After removal of the solvent in vacuo, the crude residue was purified by silica gel column chromatography to give the subtitle compound as colourless oil (0.380 g, 0.707 mmol, 71%); LC-MS: m/z=537 [MH⁺] (T=2.26).

### (iii) (S)-1-(4-{[2-Amino-4-(1-hydroxyhexan-3-ylamino)-6-methylpyrimidin-5-yl]methyl}-3-methoxyphenyl)-4-methylpiperazin-2-one

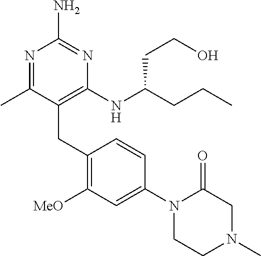

To a solution of the product from step (ii) (126 mg, 0.240 mmol) in 1,4-dioxane (1 mL) was added CuI (46.0 mg, 0.240 mmol), N,N′-dimethyldiaminoethane (52.0 μL, 0.480 mmol), 4-methylpiperazin-2-one (55.0 mg, 0.480 mmol), and Cs₂CO₃ (234 mg, 0.720 mmol). The mixture was heated to 100° C. and stirred for 10 h. After cooling, water was added and the resulting mixture was extracted with EtOAc. The combined organic solutions were washed with brine, and then dried (Na₂SO₄). After removal of the solvent in vacuo, the crude residue was used for the next reaction without further purification. To the crude residue in THF (1.0 mL) was added tetra-n-butylammonium fluoride (1.0 mL, 1M solution in THF) and the mixture was stirred at r.t. After 5 h, water was added and the resulting mixture was extracted with EtOAc. The combined organic solutions were washed with brine, and then dried (Na₂SO₄). After removal of the solvent in vacuo, the crude residue was purified by silica gel column chromatography to give the title compound as a white amorphous solid (19.6 mg, 0.0430 mmol, 18%); ¹H NMR: 6.94 (1H, d), 6.86 (1H, d), 6.75 (1H, dd), 5.07 (2H, br s), 4.90 (1H, d), 4.51 (1H, br s), 4.13 (1H, m), 3.88 (3H, s), 3.69-3.65 (2H, m), 3.67 (2H, s,), 3.31-3.26 (2H, m), 3.25 (2H, s), 2.78-2.76 (2H, m), 2.40 (3H, s), 2.35 (3H, s), 1.80-1.76 (1H, m), 1.44-1.11 (5H, m), 0.81 (3H, t); LC-MS: m/z=457 [MH⁺] (T=1.48).

### Example 3

### Alternative Method of Preparation: (S)-1-(4-{[2-Amino-4-(1-hydroxyhexan-3-ylamino)-6-methylpyrimidin-5-yl]methyl}-3-methoxyphenyl)-4-methylpiperazin-2-one

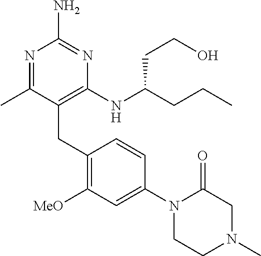

The title compound may be prepared by the following steps:

### (i) 2-Methoxy-4-(4-methyl-2-oxopiperazin-1-yl)benzaldehyde

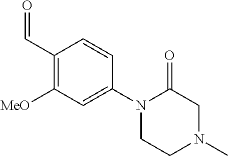 [SMILES: COc1cc(N2CCN(C)CC2=O)ccc1C=O]

To a solution of 4-bromo-2-methoxybezaldehyde (10.0 g, 46.5 mmol) in 1,4-dioxane (140 mL) was added CuI (8.84 g, 46.5 mmol), N,N′-dimethyldiaminoethane (10.0 mL, 93.0 mmol), 4-methylpiperazin-2-one (7.95 g, 69.8 mmol), and Cs₂CO₃ (45.0 g, 139 mmol). The mixture was heated to 100° C. and stirred for 5 h. After cooling, the mixture was filtered, and the solution was adjusted to pH 2-3 with 1N HCl. After stirring for 3 h at r.t., the mixture was neutralized with sat. aq. NaHCO₃, and the resulting mixture was extracted with EtOAc. The combined organic solutions were washed with brine, and then dried (Na₂SO₄). After removal of the solvent in vacuo, the subtitle compound was obtained as a white solid (10.7 g, 43.1 mmol, 93%); ¹H NMR: 10.4 (1H, s), 7.86 (1H, d), 7.11 (1H, d), 6.92 (1H, dd), 3.92 (3H, s), 3.76 (2H, t), 3.30 (2H, s), 2.82 (2H, t), 2.42 (3H, s); LC-MS: m/z=249 [MH⁺] (T=0.92 min).

### (ii) Methyl 2-(2-methoxy-4-(4-methyl-2-oxopiperazin-1-yl)benzylidene)-3-oxobutanoate

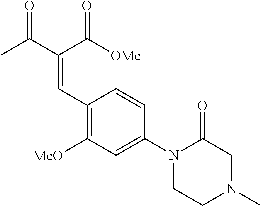 [SMILES: COC(=O)/C(=C\c1ccc(N2CCN(C)CC2=O)cc1OC)C(C)=O]

To a solution of the product from step (i) (10.7 g, 43.1 mmol) in toluene was added methyl acetylacetate (5.90 g, 51.0 mmol), acetic acid (0.980 mL, 17.2 mmol), and piperidine (0.430 mL, 4.31 mmol). The resulting mixture was heated at reflux for 20 h. After cooling, sat. aq. NaHCO₃ was added and the resulting mixture was extracted with EtOAc. The combined organic solutions were washed with brine, and then dried (Na₂SO₄). After removal of the solvent in vacuo, the crude residue was purified by silica gel column chromatography to give the subtitle compound [mixture of (E)- and (Z)-isomers] as a pale yellow oil (14.0 g, 40.5 mmol, 94%); LC-MS: m/z=347 [MH⁺] (T=2.80 min).

### (iii) Methyl 2-(2-methoxy-4-(4-methyl-2-oxopiperazin-1-yl)benzyl)-3-oxobutanoate

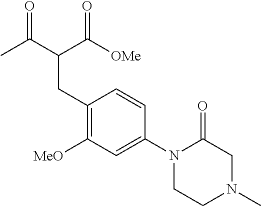 [SMILES: COC(=O)C(Cc1ccc(N2CCN(C)CC2=O)cc1OC)C(C)=O]

To a solution of the product from step (ii) (14.0 g, 40.5 mmol) in methanol (300 mL) was added 10% Pd—C (4.0 g) and stirred under H₂ (1 atm) at r.t., After 12 h, The reaction mixture was filtered through diatomaceous earth (Celite™) and the solvent was removed by evaporation to give the subtitle compound as a pale yellow oil (14.0 g, 39.2 mmol, 99%). LC-MS: m/z=349 [MH⁺] (T=2.84 min).

### (iv) 1-(4-((2-Amino-4-hydroxy-6-methylpyrimidin-5-yl)methyl)-3-methoxyphenyl)-4-methylpiperazin-2-one

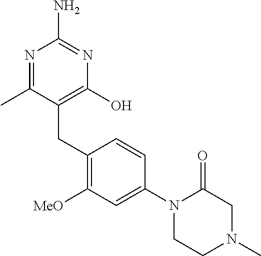

To a solution of the product from step (iii) (14.0 g, 39.2 mmol) in methanol (100 mL) was added guanidine carbonate (9.90 g, 55.0 mmol). The mixture was stirred for 6 h at reflux temperature. After cooling to r.t., the mixture was filtered, and the solvent was removed by evaporation. The crude residue was purified by silica gel column chromatography to give the subtitle compound as a white solid (6.16 g, 17.3 mmol, 43%); ¹H NMR: 6.94 (1H, d), 6.71 (1H, s), 6.66 (1H, d), 3.79 (3H, s), 3.65 (4H, m), 3.25 (2H, s), 2.77 (2H, t), 2.40 (3H, s), 1.98 (3H, s); LC-MS: m/z=358 [MH⁺] (T=0.40 min).

### (v) 2-Amino-5-(2-methoxy-4-(4-methyl-2-oxopiperazin-1-yl)benzyl)-6-methylpyrimidin-4-yl 2,4,6-trimethylbenzenesulfonate

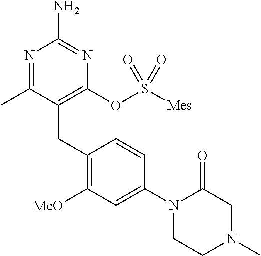

To a stirred solution of the product from step (iv) (2.50 g, 7.00 mmol) in THF (30 mL) at r.t. was added N,N,N′,N′-tetramethyl-1,3-propanediamine (1.75 mL, 10.5 mmol) and 2-mesitylenesulfonyl chloride (2.30 g, 10.5 mmol). After stirring for 12 h, water was added and the resulting mixture was extracted with EtOAc. The combined organic solutions were washed with brine, and then dried (Na₂SO₄). After removal of the solvent in vacuo, the crude residue was purified by silica gel column chromatography to give the subtitle compound as a white amorphous solid (3.60 g, 6.68 mmol, 95%); ¹H NMR: 6.95 (2H, s), 6.85 (1H, d), 6.79 (1H, d), 6.68 (1H, dd), 3.80 (5H, s), 3.70 (2H, m), 3.30 (2H, s), 2.82 (2H, m), 2.58 (6H, s), 2.43 (3H, s), 2.31 (3H, s), 2.29 (3H, s); LC-MS: m/z=540 [MH⁺] (T=3.44 min).

### (vi) (S)-1-(4-{[2-Amino-4-(1-hydroxyhexan-3-ylamino)-6-methylpyrimidin-5-yl]methyl}-3-methoxyphenyl)-4-methylpiperazin-2-one

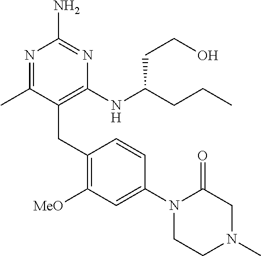

To a solution of the product from step (v) (1.90 g, 3.53 mmol) in propionitrile was added (S)-3-aminohexan-1-ol (1.23 g, 10.5 mmol) and trifluoroacetic acid (0.259 mL, 3.5 mmol). The mixture was heated to 110° C. and stirred for 9 h. After cooling to r.t., sat aq. NaHCO₃ was added and the resulting mixture was extracted with EtOAc. The combined organic solutions were washed with brine, and then dried (Na₂SO₄). After removal of the solvent in vacuo, the crude residue was purified by silica gel column chromatography to give the title compound as a white amorphous solid (1.10 g, 2.41 mmol, 68%); ¹H NMR: 6.94 (1H, d), 6.86 (1H, d), 6.75 (1H, dd), 5.07 (2H, br s), 4.90 (1H, d), 4.51 (1H, br s), 4.13 (1H, m), 3.88 (3H, s), 3.69-3.65 (2H, m), 3.67 (2H, s,), 3.31-3.26 (2H, m), 3.25 (2H, s), 2.78-2.76 (2H, m), 2.40 (3H, s), 2.35 (3H, s), 1.80-1.76 (1H, m), 1.44-1.11 (5H, m), 0.81 (3H, t); LC-MS: m/z=457 [MH⁺] (T=1.48).

### Example 4

### (S)-1-(4-{[2-Amino-4-(1-hydroxypentan-2-ylamino)-6-methylpyrimidin-5-yl]methyl}-3-methoxyphenyl)-4-methylpiperazin-2-one

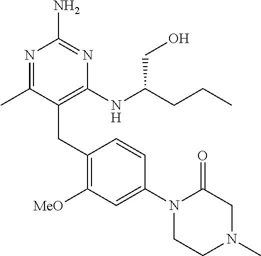

The title compound may be prepared by the steps described below:

### (i) (S)-2-[2-Amino-5-(4-bromo-2-methoxybenzyl)-6-methylpyrimidin-4-ylamino]pentan-1-ol

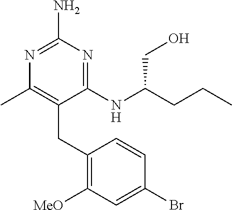 [SMILES: CCC[C@@H](CO)Nc1nc(N)nc(C)c1Cc1ccc(Br)cc1OC]

The subtitle compound was prepared using the product from Example 1 step (iii) (1.00 g, 1.98 mmol) and the method of Example 1 step (iv), in which (S)-2-aminopentan-1-ol (1.02 g, 9.90 mmol) was used instead of butylamine to give the subtitle compound as a colourless oil (0.790 g, 1.93 mmol, 98%); LC-MS: m/z=410 [MH⁺] (T=1.81).

### (ii) (S)-5-(4-Bromo-2-methoxybenzyl)-N⁴-[1-(tert-butyldimethylsilyloxy)pentan-2-yl]-6-methylpyrimidine-2,4-diamine

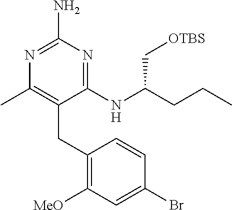 [SMILES: CCC[C@@H](CO[Si](C)(C)C(C)(C)C)Nc1nc(N)nc(C)c1Cc1ccc(Br)cc1OC]

The subtitle compound was prepared by the method of Example 3 step (ii) using the product from step (i) (131 mg, 0.320 mmol) to give the subtitle compound as a colourless oil (27 mg, 0.0517 mmol, 16%); LC-MS: m/z=523 [MH⁺] (T=2.15).

### (iii) (S)-1-(4-{[2-Amino-4-(1-hydroxypentan-2-ylamino)-6-methylpyrimidin-5-yl]methyl}-3-methoxyphenyl)-4-methylpiperazin-2-one

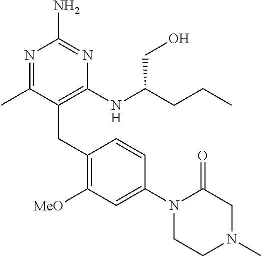

The title compound was prepared by the method of Example 3 step (iii) using the product from step (ii) (27.0 mg, 0.0517 mmol) to give the title compound as a colourless oil (5.70 mg, 0.0129 mmol, 25%); ¹H NMR: 6.98 (1H, d), 6.82 (1H, d), 6.75 (1H, dd), 5.84 (2H, br s), 5.58 (1H, d), 4.12 (1H, m), 3.88 (3H, s), 3.74 (2H, s,), 3.73-3.65 (2H, m), 3.45-3.40 (2H, m), 3.25 (2H, s), 2.81-2.77 (2H, m), 2.46 (3H, s), 2.40 (3H, s), 1.47-1.20 (4H, m), 0.86 (3H, t); LC-MS: m/z=443 [MH⁺] (T=1.35).

### Example 5

### (S)-1-(4-{[2-Amino-4-(1-hydroxyhexan-3-ylamino)-6-methylpyrimidin-5-yl]methyl}-3-methoxyphenyl)piperazin-2-one

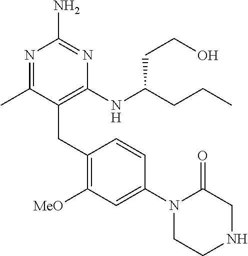 [SMILES: CCC[C@@H](CCO)Nc1nc(N)nc(C)c1Cc1ccc(N2CCNCC2=O)cc1OC]

The title compound may be prepared by the steps described below:

### (i) (S)-tert-butyl 4-(4-((2-amino-4-(1-hydroxyhexan-3-ylamino)-6-methylpyrimidin-5-yl)methyl)-3-methoxyphenyl)-3-oxopiperazine-1-carboxylate

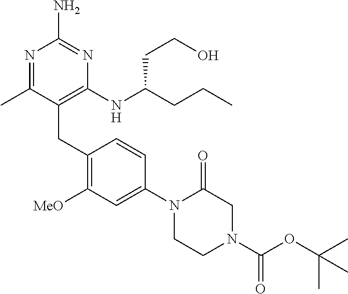 [SMILES: CCC[C@@H](CCO)Nc1nc(N)nc(C)c1Cc1ccc(N2CCN(C(=O)OC(C)(C)C)CC2=O)cc1OC]

The subtitle compound was prepared using the product from Example 3 step (ii) (80.0 mg, 0.149 mmol) and the method of Example 3 step (iii), in which tert-butyl 3-oxopiperazine-1-carboxylate (40.0 mg, 0.200 mmol) was used instead of 4-methylpiperazin-2-one to give the subtitle compound as a colourless oil (70 mg, 0.129 mmol, 86%); ¹H NMR: 6.95 (1H, d), 6.86 (1H, d), 6.74 (1H, dd), 4.91 (2H, br s), 4.54 (1H, br s), 4.24 (2H, s), 4.13 (1H, m), 3.90 (3H, s), 3.80-3.67 (6H, m), 3.33 (2H, m), 2.34 (3H, s), 1.81-1.11 (6H, m), 1.50 (9H, s), 0.81 (3H, t); LC-MS: m/z=543 [MH⁺] (T=0.74)

### (ii) (S)-1-(4-{[2-Amino-4-(1-hydroxyhexan-3-ylamino)-6-methylpyrimidin-5-yl]methyl}-3-methoxyphenyl)piperazin-2-one

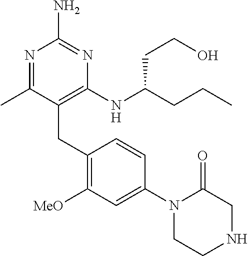 [SMILES: CCC[C@@H](CCO)Nc1nc(N)nc(C)c1Cc1ccc(N2CCNCC2=O)cc1OC]

To a solution of the product from step (i) (67 mg, 0.124 mmol) in chloroform (0.8 mL) was added hydrogen chloride (0.8 mL, 4 M solution in 1,4-dioxane) and the mixture was stirred at r.t. After 3 h, 10% aq. K₂CO₃ was added, and the resulting mixture was extracted with EtOAc. The combined organic solutions were washed with brine, and then dried (Na₂SO₄). After removal of the solvent in vacuo, the crude residue was purified by silica gel column chromatography to give the title compound as a colourless oil (22.0 mg, 0.0498 mmol, 40%); ¹H NMR: 6.94 (1H, d), 6.87 (1H, d), 6.75 (1H, dd), 4.90 (1H, br s), 4.67 (2H, br s), 4.55 (1H, d), 4.11 (1H, m), 3.89 (3H, s), 3.68-3.60 (6H, m), 3.40-3.19 (4H, m), 2.30 (3H, s), 1.91-1.77 (1H, m), 1.44-1.09 (5H, m), 0.79 (3H, t); LC-MS: m/z=443 [MH⁺] (T=0.44)

### Biological Assays

The ability of compounds to activate TLR7 in vitro was assessed using the human TLR7 assay described below.

### Human TLR7 Assay

Recombinant human TLR7 was stably expressed in a HEK293 cell line already stably expressing the pNiFty2-SEAP reporter plasmid; integration of the reporter gene was maintained by selection with the antibiotic zeocin. The most common variant sequence of human TLR7 (represented by the EMBL sequence AF240467) was cloned into the mammalian cell expression vector pUNO and transfected into this reporter cell-line. Transfectants with stable expression were selected using the antibiotic blasticidin. In this reporter cell-line, expression of secreted alkaline phosphatase (SEAP) is controlled by an NF-κB/ELAM-1 composite promoter comprising five NF-κB sites combined with the proximal ELAM-1 promoter. TLR signaling leads to the translocation of NF-κB and activation of the promoter results in expression of the SEAP gene. TLR7-specific activation was assessed by determining the level of SEAP produced following overnight incubation of the cells at 37° C. with the standard compound in the presence of 0.1% (v/v) dimethylsulfoxide (DMSO). Concentration dependent induction of SEAP production by compounds was expressed as the concentration of compound which produced half of the maximal level of SEAP induction for that compound (EC₅₀). TLR7 activity for compounds of the present invention was assessed using the human TLR7 assay and the results are shown in Table 1 below wherein the degree of TLR7 activation for each compound is expressed as a pEC₅₀ value.

TABLE 1

| Ex. No. | pEC₅₀ |
|---|---|
| 1 | 7.2 |
| 2 | 7.2 |
| 3 | 7.6 |
| 4 | 6.7 |
| 5 | 6.9 |

### Human TLR8 Assay

TLR8/NF-κB/SEAPorter™ HEK 293 Cell Line (Imgenex Corporation) is a stably co-transfected cell line which expresses full-length human TLR8 and the secreted alkaline phosphatase (SEAP) reporter gene under the transcriptional control of an NF-κB response element. TLR8 expression in this cell line has been tested by flow cytometry. Transfectants with stable expression were selected using the antibiotic blasticidin and geneticin. TLR signaling leads to the translocation of NF-κB and activation of the promoter results in expression of the SEAP gene. TLR8-specific activation was assessed by determining the level of SEAP produced following overnight incubation of the cells at 37° C. with the standard compound in the presence of 0.1% (v/v) dimethylsulfoxide (DMSO). Concentration dependent induction of SEAP production by compounds was expressed as the concentration of compound which produced half of the maximal level of SEAP induction for that compound (EC₅₀). TLR8 activity for compounds of the present invention was assessed using the human TLR8 assay and the results are shown in Table 2 below wherein the degree of TLR8 activation for each compound is expressed as a pEC₅₀ value.

TABLE 2

| Ex. No. | pEC₅₀ |
|---|---|
| 1 | <5 |
| 2 | <5 |
| 3 | 5.2 |
| 4 | 5.3 |
| 5 | <5 |

hERG (Human Ether-a-go-go Related Gene) Analysis—Method 1

The hERG potassium current is measured in a hERG-stably-expressing Chinese hamster ovary K1 (CHO) cells. The experiments were performed using an automated planar patch-clamp system QPatch HT (Sophion Bioscience A/S). The application of pressure for forming gigaseals and whole-cell patch clamp configuration were established using the QPatch assay software. Patch-clamp experiments were performed in voltage-clamp mode and whole-cell currents were recorded from individual cells. The following stimulation protocol was applied to investigate the effects of compounds on hERG potassium channel.: The membrane potential was held at −80 mV and repetitively (every 15 s) depolarized to +20 mV for 5 s after the pulse to −50 mV for 20 ms served to define the baseline, followed by repolarizing step to −50 mV for 5 s to evaluate of the tail current amplitude. Experiments were conducted at room temperature (22±2° C.).

Effects of compounds were determined from cumulative applications of increasing 4 concentrations and calculated as percent of blocked current. The data points were fitted with Hill equation to calculate half-maximal inhibition concentrations.

The test solution includes:

Extracellular solution (mM): 2 mM of CaCl₂, 1 mM of MgCl₂, 10 mM of HEPES, 4 mM of KCl, 145 mM of NaCl, and 10 mM of glucose; and

Intracellular solution (mM): 5.4 mM of CaCl₂, 1.8 mM of MgCl₂, 10 mM of HEPES, 31 mM of KOH, 10 mM of EGTA, 120 mM of KCl, and 4 mM of ATP.

The results are shown in Table 3, below.

hERG Analysis—Method 2

### Cell Culture

The hERG-expressing Chinese hamster ovary K1 (CHO) cells described by (Persson, Carlsson, Duker, & Jacobson, 2005) are grown to semi-confluence at 37° C. in a humidified environment (5% CO₂) in F-12 Ham medium containing L-glutamine, 10% foetal calf serum (FCS) and 0.6 mg/mL hygromycin (all available from Sigma-Aldrich). Prior to use, the monolayer is washed using a pre-warmed (37° C.) 3 mL aliquot of Versene 1:5,000 (Invitrogen). After aspiration of this solution the flask is incubated at 37° C. in an incubator with a further 2 mL of Versene 1:5,000 for a period of 6 minutes. Cells are then detached from the bottom of the flask by gentle tapping and 10 mL of Dulbecco's Phosphate-Buffered Saline containing calcium (0.9 mM) and magnesium (0.5 mM) (PBS; Invitrogen) is then added to the flask and aspirated into a 15 mL centrifuge tube prior to centrifugation (50 g, for 4 mins). The resulting supernatant is discarded and the pellet gently re-suspended in 3 mL of PBS. A 0.5 mL aliquot of cell suspension is removed and the number of viable cells (based on trypan blue exclusion) is determined in an automated reader (Cedex; Innovatis) so that the cell re-suspension volume can be adjusted with PBS to give the desired final cell concentration. It is the cell concentration at this point in the assay that is quoted when referring to this parameter. CHO-Kv1.5 cells, which are used to adjust the voltage offset on IonWorks™ HT, are maintained and prepared for use in the same way.

### Electrophysiology

The principles and operation of this device have been described by (Schroeder, Neagle, Trezise, & Worley, 2003). Briefly, the technology is based on a 384-well plate (PatchPlate™) in which a recording is attempted in each well by using suction to position and hold a cell on a small hole separating two isolated fluid chambers. Once sealing has taken place, the solution on the underside of the PatchPlate™ is changed to one containing amphotericin B. This permeablises the patch of cell membrane covering the hole in each well and, in effect, allows a perforated, whole-cell patch clamp recording to be made.

A β-test IonWorks™ HT from Essen Instrument was used. There is no capability to warm solutions in this device hence it is operated at ˜r.t. (˜21° C.), as follows. The reservoir in the “Buffer” position is loaded with 4 mL of PBS and that in the “Cells” position with the CHO-hERG cell suspension described above. A 96-well plate (V-bottom, Greiner Bio-one) containing the compounds to be tested (at 3-fold above their final test concentration) is placed in the “Plate 1” position and a PatchPlate™ is clamped into the PatchPlate™ station. Each compound plate is laid-out in 12 columns to enable ten, 8-point concentration-effect curves to be constructed; the remaining two columns on the plate are taken up with vehicle (final concentration 0.33% DMSO), to define the assay baseline, and a supra-maximal blocking concentration of cisapride (final concentration 10 μM) to define the 100% inhibition level. The fluidics-head (F-Head) of IonWorks™ HT then adds 3.54 of PBS to each well of the PatchPlate™ and its underside is perfused with “internal” solution that had the following composition (in mM): K-Gluconate (100 parts), KCl (40 parts), MgCl₂ (3.2 parts), EGTA (3 parts) and HEPES (5 parts, pH 7.25-7.30 using 10M KOH). After priming and de-bubbling, the electronics-head (E-head) then moves round the PatchPlate™ performing a hole test (i.e. applying a voltage pulse to determine whether the hole in each well is open). The F-head then dispenses 3.54 of the cell suspension described above into each well of the PatchPlate™ and the cells are given 200 seconds to reach and seal to the hole in each well. Following this, the E-head moves round the PatchPlate™ to determine the seal resistance obtained in each well. Next, the solution on the underside of the PatchPlate™ is changed to “access” solution that has the following composition (in mM): KCl (140 parts), EGTA (1 part), MgCl₂ (1 part) and HEPES (20 parts, pH 7.25-7.30 using 10M KOH) plus 100 μg/mL of amphotericin B (Sigma-Aldrich). After allowing 9 minutes for patch perforation to take place, the E-head moves round the PatchPlate™ 48 wells at a time to obtain pre-compound hERG current measurements. The F-head then adds 3.54 of solution from each well of the compound plate to 4 wells on the PatchPlate™ (the final DMSO concentration is 0.33% in every well). This is achieved by moving from the most dilute to the most concentrated well of the compound plate to minimise the impact of any compound carry-over. After approximately 3.5 mins incubation, the E-head then moves around all 384-wells of the PatchPlate™ to obtain post-compound hERG current measurements. In this way, non-cumulative concentration-effect curves can be produced where, providing the acceptance criteria are achieved in a sufficient percentage of wells (see below), the effect of each concentration of test compound is based on recording from between 1 and 4 cells.

The pre- and post-compound hERG current is evoked by a single voltage pulse consisting of a 20 second period holding at −70 mV, a 160 millisecond step to −60 mV (to obtain an estimate of leak), a 100 millisecond step back to −70 mV, a 1 second step to +40 mV, a 2 second step to −30 mV and finally a 500 millisecond step to −70 mV. In between the pre- and post-compound voltage pulses there is no clamping of the membrane potential. Currents are leak-subtracted based on the estimate of current evoked during the +10 mV step at the start of the voltage pulse protocol. Any voltage offsets in IonWorks™ HT were adjusted in one of two ways. When determining compound potency, a depolarising voltage ramp is applied to CHO-Kv1.5 cells and the voltage noted at which there was an inflection point in the current trace (i.e. the point at which channel activation is seen with a ramp protocol). The voltage at which this occurred has previously been determined using the same voltage command in conventional electrophysiology and found to be −15 mV (data not shown); thus an offset potential could be entered into the IonWorks™ HT software using this value as a reference point. When determining the basic electrophysiological properties of hERG, any offset is adjusted by determining the hERG tail current reversal potential in IonWorks™ HT, comparing it with that found in conventional electrophysiology (−82 mV) and then making the necessary offset adjustment in the IonWorks™ HT software. The current signal is sampled at 2.5 kHz.

Pre- and post-scan hERG current magnitude was measured automatically from the leak subtracted traces by the IonWorks™ HT software by taking a 40 ms average of the current during the initial holding period at −70 mV (baseline current) and subtracting this from the peak of the tail current response. The acceptance criteria for the currents evoked in each well are: pre-scan seal resistance >60MΩ, pre-scan hERG tail current amplitude >150 pA; post-scan seal resistance >60MΩ. The degree of inhibition of the hERG current can be assessed by dividing the post-scan hERG current by the respective pre-scan hERG current for each well. References: Persson, F. et al, J. Cardiovasc. Electrophysiol., 16, 329-341 (2005), and Schroeder, K., et al, J Biomol Screen., 8, 50-64, (2003).

The results are shown in Table 3.

TABLE 3

| Ex. No. | IC₅₀ Method 1 | IC₅₀ Method 2 |
|---|---|---|
| 1 | 2.9 | NT |
| 2 | 0.80 | 5.41 |
| 3 | >10 | >33 |
| 4 | >10 | NT |
| 5 | >10 | NT |

### PAMPA Analysis

### Preparation of Test Solution

Donor: System Solution Concentrate (pION inc.) (25 mL) and DMSO (50 mL) (f.c. 5%) was diluted to 1 L with milliQ water, adjusted to pH5.0 or 7.4 with NaOH. Compound solution (5 μL, 10 mM solution in DMSO) was added to 1 mL of system solution prepared as above, and filtered.

### Acceptor: Acceptor Sink Buffer(pION inc.)

### Permeability Experiment

Compound solution (200 μL) was added to Donor plate. GIT Lipid-0 (pION inc.) (44) was added to Acceptor plate. Acceptor Sink Buffer (200 μL) was added to Acceptor plate. Donor and Acceptor plates were superposed. After incubation for 4 h under humidified conditions, the concentration of test compound in both solutions of Acceptor and Donor plates were measured by UV plate reader (190-500 nm).
Permeation coefficient Pe(10⁻⁶ cm/sec) was calculated by the equation shown below.

$${P}_{e} = - \frac{2.303 {V}_{D}}{A ( t - {τ}_{SS} )} ( \frac{1}{1 + {r}_{a}} ) \cdot {log}_{10} [ - {r}_{a} + ( \frac{1 + {r}_{a}}{1 - R} ) \cdot \frac{{C}_{D} ( t )}{{C}_{D} ( 0 )} ]$$

rₐ=(V_{D}/V_{A})Pₑ^{(A→D)}/Pₑ^{(D→A)}=r_{V}Pₑ^{(A→D)}/Pₑ^{(D→A)}

r_{V}=(V_{D}/V_{A})
V_{D}=volume of donor well
V_{A}=volume of acceptor well
t=permeation time
τ^{SS}=steady state time
R=retention
C_{D} and C_{A}=concentration in donor and acceptor well
   The results are shown in Table 4.

TABLE 4

| Ex. No. | Pₑ(10⁻⁶ cm/sec) pH 7.4 |
|---|---|
| 1 | 35.6 |
| 2 | 44.2 |
| 3 | 23.7 |
| 4 | 1.3 |
| 5 | <0.1 |

1. A compound of Formula (I), or a pharmaceutically acceptable salt thereof: 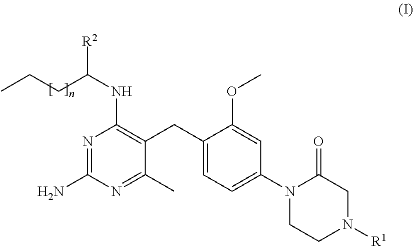
wherein:
n is 1 or 2;
R¹ is selected from hydrogen, C₁₋₄alkyl, C₁₋₃alkoxyC₂₋₄alkyl, hydroxyC₂₋₄alkyl and
(R^{X})(R^{Y})N—C₂₋₄alkyl, wherein R^{X} and R^{Y} each independently represent hydrogen or C₁₋₃alkyl; and
R² is hydrogen, hydroxymethyl or 2-hydroxyethyl.

2. A compound according to claim 1, or a pharmaceutically acceptable salt thereof, wherein n is 1.

3. A compound according to claim 1 or claim 2, or a pharmaceutically acceptable salt thereof, wherein R¹ is hydrogen or C₁₋₄alkyl.

4. A compound according to any one of claims 1 to 3, or a pharmaceutically acceptable salt thereof, wherein R¹ is methyl.

5. A compound according to claim 1 selected from a group consisting of:
1-(4-{[2-Amino-4-(butylamino)-6-methylpyrimidin-5-yl]methyl}-3-methoxyphenyl)-4-methylpiperazin-2-one;
1-(4-{[2-Amino-4-methyl-6-(pentylamino)pyrimidin-5-yl]methyl}-3-methoxyphenyl)-4-methylpiperazin-2-one;
(S)-1-(4-{[2-Amino-4-(1-hydroxyhexan-3-ylamino)-6-methylpyrimidin-5-yl]methyl}-3-methoxyphenyl)-4-methylpiperazin-2-one;
(S)-1-(4-{[2-Amino-4-(1-hydroxypentan-2-ylamino)-6-methylpyrimidin-5-yl]methyl}-3-methoxyphenyl)-4-methylpiperazin-2-one;
(S)-1-(4-{[2-Amino-4-(1-hydroxyhexan-3-ylamino)-6-methylpyrimidin-5-yl]methyl}-3-methoxyphenyl)piperazin-2-one;
or a pharmaceutically acceptable salt thereof.

6. A pharmaceutical composition which comprises a compound according to any one of claims 1 to 5, or a pharmaceutically acceptable salt thereof, in association with a pharmaceutically acceptable diluent or carrier.

7. A compound according to any one of claims 1 to 5, or a pharmaceutically acceptable salt thereof, for use as a medicament.

8. A compound according to any one of claims 1 to 5, or a pharmaceutically acceptable salt thereof, for use in the treatment of cancer.

9. Use of a compound according to any one of claims 1 to 5, or a pharmaceutically acceptable salt thereof, in the manufacture of a medicament for use in the treatment of cancer.

10. A method of treating cancer in a warm blooded animal, such as man, in need of such treatment which comprises administering to said animal an effective amount of a compound of the Formula (I), or a pharmaceutically acceptable salt thereof, as defined in any one of claims 1 to 5.

11. A process for the preparation of a compound of Formula (I), or a pharmaceutically acceptable salt thereof, as defined in claim 1, which comprises a step of reacting a compound of Formula (XXIII): 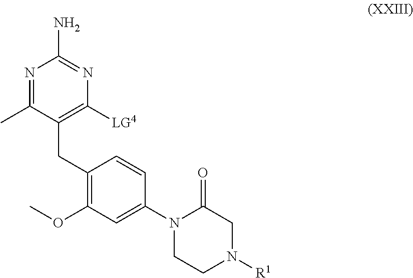
wherein R¹ is as defined in Formula (I) in claim 1, and LG⁴ is a leaving group;
with a compound of Formula (XII): 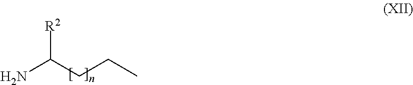
wherein R² and n are as defined in Formula (I) in claim 1, in the presence or absence of a base.
